(19) United States
(12) Patent Application Publication
(10) Pub. No.: US 2011/0039280 A1
(43) Pub. Date: Feb. 17, 2011

(54) HYBRID MICROFLUIDIC SPR AND MOLECULAR IMAGING DEVICE

(75) Inventors: James F. Leary, West Lafayette, IN (US); Kinam Park, West Lafayette, IN (US); Ghanashyam Acharya, West Lafayette, IN (US); Michael Zordan, Carmel, IN (US)

Correspondence Address: BRINKS HOFER GILSON & LIONE, P.O. BOX 10395, CHICAGO, IL, 60610, US

(73) Assignee: PURDUE RESEARCH FOUNDATION, West Lafayette, IN (US)

(21) Appl. No.: 12/740,087

(22) PCT Filed: Oct. 29, 2008
(86) PCT No.: PCT/US2008/081571

§ 371 Date: Sep. 2, 2010

Related U.S. Application Data
(60) Provisional application No. 61/093,035, filed on Aug. 29, 2008.
(60) Provisional application No. 60/983,412, filed on Oct. 29, 2007.

(51) Int. Cl. G01N 33/569; C12M 1/34

(52) U.S. Cl. 435/7.32; 435/288.7

(57) ABSTRACT

A hybrid microfluidic biochip designed to perform multiplexed detection of singled- celled pathogens using a combination of SPR and epi-fluorescence imaging. The device comprises an array of gold spots, each functionalized with a capture biomolecule targeting a specific pathogen. This biosensor array is enclosed by a polydimethylsiloxane (PDMS) microfluidic flow chamber that delivers a magnetically concentrated sample to be tested. The sample is imaged by surface plasmon resonance on the bottom of the biochip, and epi- fluorescence on the top.

### CROSS-REFERENCE TO RELATED APPLICATIONS

This application is based on, and claims benefit to U.S. Provisional Applications 61/093,035, filed on Aug. 29, 2008, and 60/983,412, filed on Oct. 29, 2007, both which are incorporated herein by reference.

### GOVERNMENT INTERESTS

The U.S. Government has a paid-up license in this invention and the right in limited circumstances to require the patent owner to license others on reasonable terms as provided for by the terms of Grant No. 58-1935-4-430 awarded by the U.S. Department of Agriculture.

### TECHNICAL FIELD

The present disclosure relates generally to systems for the detection of biological agents, and more specifically, to hybrid microfluidic surface plasmon resonance (SPR) and molecular imaging systems for the detection of biological agents.

### BACKGROUND

Development of simple and specific biosensors to detect pathogenic bacteria and spores has far-reaching implications in their timely identification prior to infection, which is of great concern to human health and safety. Due to the growing antibiotic resistance and the emergence of pathogenic bacteria as either dangers to the food supply or as bioterrorism agents, continuous monitoring of the environment for infectious diseases is important. To be accepted, this continuous environmental monitoring requires the integration of simple, practical, and cost-effective methodologies into handheld field ready devices that are highly sensitive and specific. The swift and broad microbial screening scenario is, currently unable to identify microbes in the field without batteries of assays that frequently result in false positives. Many tests respond to multiple organisms. The laboratory testing, though more precise than field tests, is often excruciatingly slow. The rapid and accurate identification of pathogens is a vital task for the first responders in order to facilitate timely and appropriate actions in the event of a pathogenic outbreak either naturally in the food/water supply or deliberately caused as part of bioterrorist action.

Due to the potential of B. anthracis for use as an agent of bioterrorism, its proven record of occupational exposure, and the persistence of spores in the environment, the development of rapid and accurate detection methods is of immediate importance. The accurate and rapid diagnosis of anthrax is necessary since the infection is often difficult to diagnose, spreads rapidly, and has a high mortality rate. Compounding the threat is the fact that Anthrax being an infectious disease requires medical attention within a few hours of initial inhalation and it takes approximately 48 hours for the first symptoms to appear. Therefore, the rapid detection of B. anthracis spores in the environment prior to infection is an extremely important goal for human health and safety.

The antibody and nucleic acid based detection approaches consist of complex, multi-step, time consuming, and labor intensive assay formats and target analyte analysis to ensure the specificity of detection. The currently available detection methods are of considerable importance in medical diagnostics and epidemiology, but they are not suitable for the rapid pathogen detection for preventing exposure as they are only applicable after exposure to the organisms has occurred. The drawback to these otherwise very effective immunoassays is that death normally results in patients prior to sufficient antibody levels being produced, or before a blood culture of the pathogen can be grown for detection of antibodies.

The vast majority of array-based studies of bioaffinity interactions employ fluorescently labeled biomolecules or enzyme-linked colorimetric assays. However, there is a need for methods that detect bioaffinity interactions without molecular labels, especially for biomolecular and cellular interactions, where labeling is problematic and can interfere with their biological properties.

What is needed are detection systems that are simple, rapid, accurate, and highly sensitive. Additionally, detection systems are needed that are portable and require minimal maintenance.

### SUMMARY

A system of detecting biological agents is provided. Preferably, the system comprises a pre-capture unit, a surface plasmon resonance unit, and a molecular imaging unit. More preferably, the system comprises a pre-capture unit adapted to sequester pathogens from a fluid or gas and increase pathogen concentration into a volume suitable for transfer to a microfluidic biochip unit; a microfluidic biochip unit coupled to the pre-capture unit, the microfluidic biochip having contact printed surfaces comprising pathogen-specific capture ligands adapted to capture pathogens; a surface plasmon resonance imaging unit adapted to detect the captured pathogens by surface plasmon resonance imaging; a molecular imaging unit adapted to detect the captured pathogens by epi-fluorescence imaging; and at least one small imaging camera adapted to capture surface plasmon resonance and molecular imaging data, the at least one small camera coupled to a computing device.

In one aspect, the system of detecting biological agents comprises a hybrid microfluidic biochip designed to perform multiplexed detection of single-celled pathogens using a combination of SPR and epi-fluorescence imaging.

In another aspect, the system of detecting biological agents comprises a surface plasmon resonance system that can specifically detect specific multiple pathogens rapidly in real time with high sensitivity.

In yet another aspect, the system of detecting biological agents comprises a miniaturized SPR imaging system which affords a simple, compact, inexpensive, portable SPR imaging device.

In another aspect, the system of detecting biological agents comprises a high resolution digital camera for real time imaging of pathogenic bacteria and spores that become bound to the sensor surface.

In another aspect, the system of detecting biological agents comprises a pre-capture unit adapted to capture magnetic micro- or nanoparticle labeled microbes.

In yet another aspect, the system of detecting biological agents comprises a microfluidic biochip having contact printed surfaces comprising gold.

In another aspect, the system of detecting biological agents comprises pathogen-specific capture ligands comprising peptides, antibodies, aptamers, and combinations thereof.

In another aspect, the system of detecting biological agents comprises a pre-capture unit adapted to capture magnetic micro- or nanoparticle labeled microbes coated with antibodies, peptides, aptamers, lipophilic molecules, and combinations thereof

In another aspect, a method of detecting biological agents is provided.

Other systems, methods, features and advantages will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The system may be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1A shows a multi-component schematic of the overall pathogen detection system.

FIG. 1B shows an alternative multi-component schematic of the overall pathogen detection system.

FIG. 1C shows a schematic of a portable SPR imaging hybrid imaging system with associated microfluidic chip (left). A picture of the constructed SPR imaging hybrid imaging system (right).

FIG. 2 shows pre-concentration of pathogens prior to microfluidic analysis.

FIG. 3 shows a schematic of a microfluidic chip mold design, (A) side view, (B) top view.

FIG. 4 shows a schematic of the overall microfluidic chip assembly process.

FIG. 5A shows a schematic depicting micro-contact printing of peptide arrays on a biosensor surface.

FIG. 5B shows specific peptide sequences to Bacillus subtilis (a) and Bacillus anthracis (b).

FIG. 6 shows the pattern of functionalization of the gold array (left). Gold spots were functionalized with either E. coli O157:H7 antibody, rabbit pre-immune serum, or 1% BSA. Then either E. coli O157:H7 or E. coli DH5-α were added to each spot. FIG. 6 shows a fluorescence image of the gold array demonstrating the selective capture of pathogens (right).

FIG. 7 shows the amount of gold spot surface area occupied by bound pathogen for each strain of E. coli and each surface functionalization.

FIG. 8 shows SPR images (A and C) and fluorescence images (B and D) of E. coli at high and low cell densities.

FIG. 9 shows SPR images and fluorescent molecular images of fluorescently labeled (for live/dead status of bacterial pathogens) bacteria bound to ligand-labeled contact regions on a chip.

Table 1 shows absorbance measurements of magnetic beads linked to E. coli O157:H7 at initial concentrations and reconstituted concentrations.

### DETAILED DESCRIPTION OF THE PREFERRED EMBODIMENTS

Unless otherwise defined, all technical and scientific terms used herein have the same meaning as commonly understood by one of ordinary skill in the art to which this invention pertains. In case of conflict, the present document will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

### a) Overall Design

A surface plasmon resonance imaging biosensor is disclosed for the rapid, label-free, and high throughput detection of food or water-borne pathogens. The device integrates an SPR imaging system with a biosensor array immobilized onto the sample surface containing specific biomolecules. A microfluidic chip encloses the biosensor array to administer the sample. A group of biomolecules are immobilized onto an array of gold spots on a glass slide. This biomolecule imprinted gold chip functions as a biosensor array for the specific detection of pathogens. A portable hybrid SPR/molecular imaging system is provided to determine what fraction of pathogenic bacteria are live or dead (since dead pathogenic bacteria may pose little or no threat) and to confirm SPR results. The portable hybrid SPR/molecular imaging system can also provide additional information of pathogen status, such as for example, metabolic state.

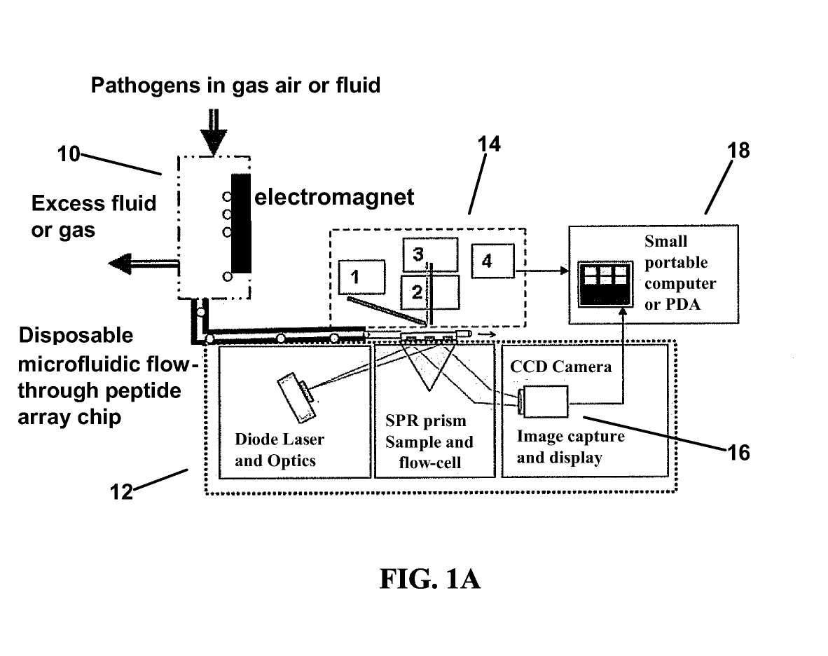

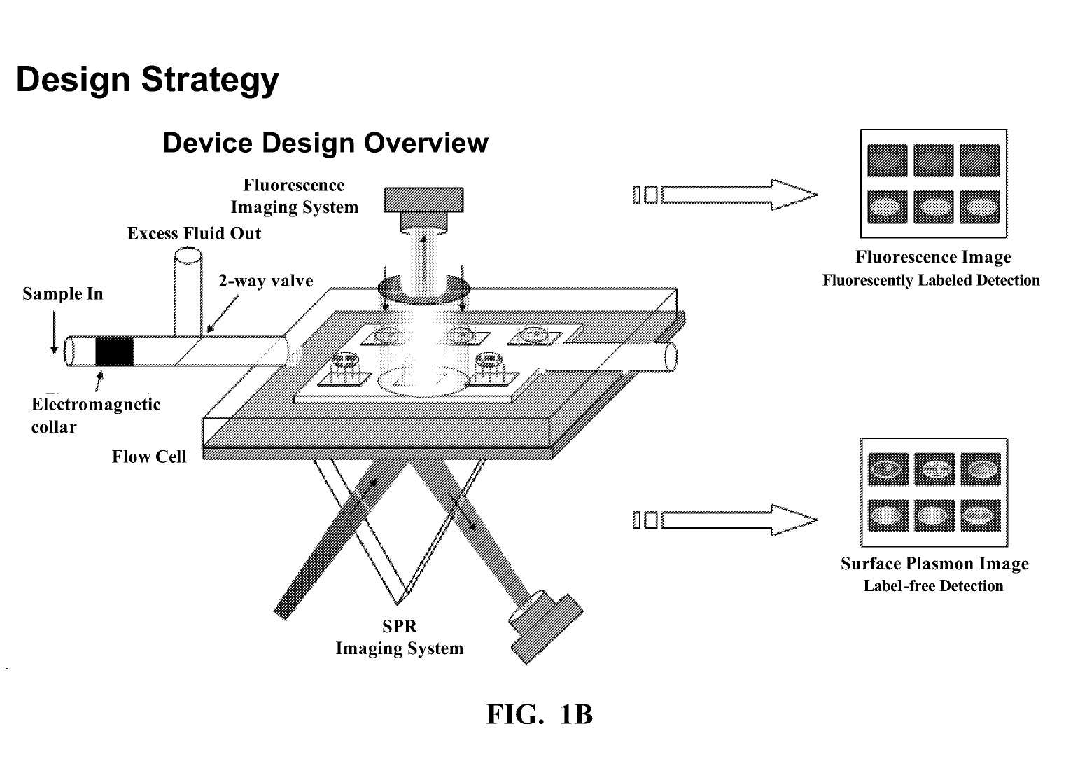

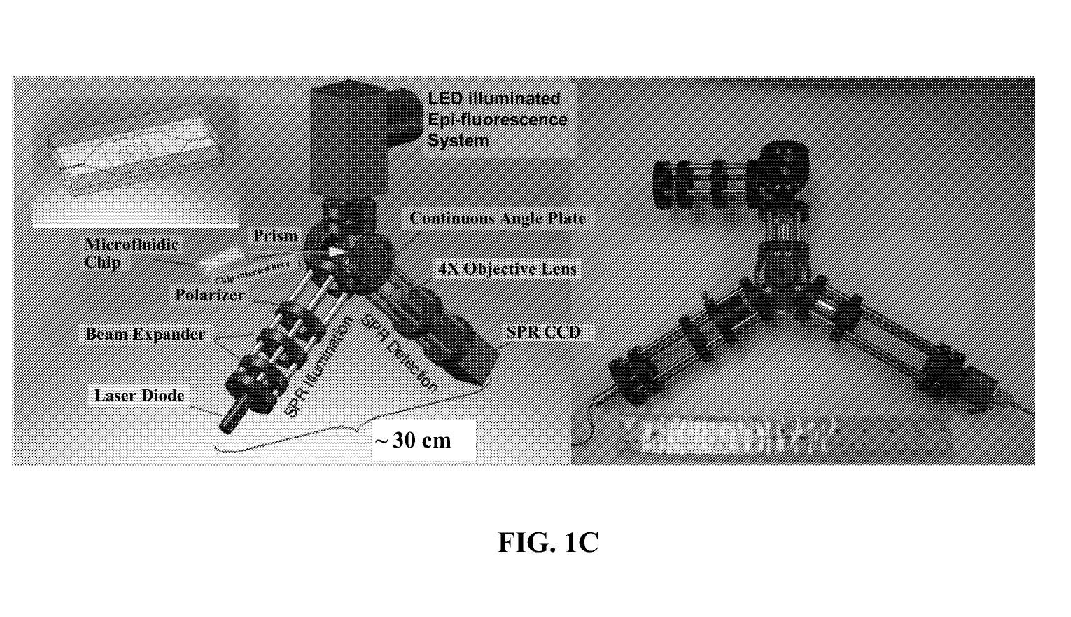

A schematic of the overall conceptual design of this portable pathogen detection system is shown in FIG. 1A and FIG. 1B. The overall instrument has three modular subsystems (pre-concentrator, molecular imaging, SPR imaging) which can be modified for more specific functions.

Preferably, this hybrid, multi-component device of FIG. 1A contains: (1) a front-end magnetic concentrator 10 to capture magnetic micro- or nanoparticle labeled microbes and increase their concentration into a smaller volume suitable for a microfluidic flow/imaging device; (2) a surface plasmon imaging subsystem 12 to detect captured microbes on a patterned grid of gold contact spots; (3) a molecular imaging epi-fluorescence subsystem 14 to determine viability and functional status of the captured microbes, the molecular imaging epi-fluorescence subsystem comprising a blue light-emitting diode 1, optical filters 2, a CCD array 3, and signal processing electronics 4; and (4) at least one small imaging camera 16 to capture imaging data, the camera coupled to a portable computing device 18 (e.g., laptop computer, PDA-type device, or the like). This computing device can contain automated image analysis and other software (implemented in Matlab executables) to do completely automated analysis for pathogen detection.

The instrument can be assembled as a bench top instrument, or alternatively, as a hand-held, portable device. FIG. 1C shows a schematic of a portable SPR imaging hybrid imaging system with associated microfluidic chip; and a picture of the constructed portable SPR imaging hybrid imaging system. The mini-optical rail system gives flexibility and structural integrity to the device so that it can be self-supporting and portable.

### b) Magnetic Pre-Concentration

Since microfluidic devices by definition can only sample small amounts of fluid, it is important to pre-concentrate all possible pathogens present in large volumes of fluid prior to microfluidic analysis. There are several ways that this can be accomplished. The method used to concentrate bacteria as described herein involves use of a specific antibody against the bacterial strain that is being screened. Use of specific antibodies, or other capture molecules such as peptides or aptamers, works well but requires specific reagents and creation of a multiplexed magnetic capture molecule system.

An alternative approach is to use magnetic nano- or micro-particles coated with lipophilic molecules. Virtually all pathogens have a lipophilic outer coating and will fuse with these coated nanoparticles. It is only necessary for one or a few nanoparticles to bind to the pathogens in order to pull them out of large volumes of water (or other fluids) or air (or other gases). All pathogens can be quickly labeled with lipophilic nanoparticles which will bind to virtually any pathogen. Then these nanoparticle labeled pathogens can be captured and held against a surface while excess fluid is discarded. When the magnetic field is removed, the captured pathogens can be flowed in much smaller volumes of fluid, more appropriate for microfluidic device analysis, across a large surface containing molecular capture ligands (e.g. antibodies, peptides, aptamers, etc.).

Regardless of the capturing approach used, the coated magnetic particles serve to pre-concentrate the pathogens into a much smaller volume enabling potentially rare pathogens to be sampled and detected in relatively large volumes. This translates to very large improvements in sampling statistics. The coated micro- or nanoparticles, if appropriately chosen, do not significantly block the accessibility of other pathogen-specific surface molecules that can be subsequently detected by flowing these concentrated pathogens across contact printed surfaces labeled with pathogen-specific binding peptides, antibodies or other ligands.

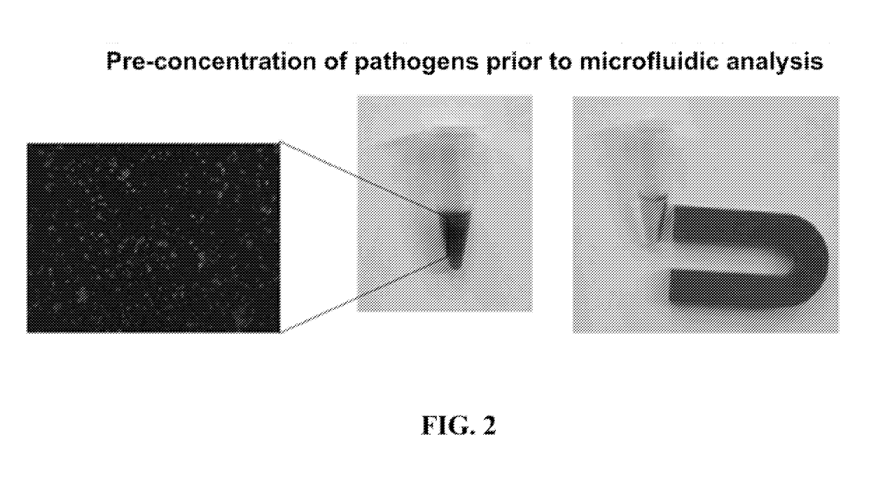

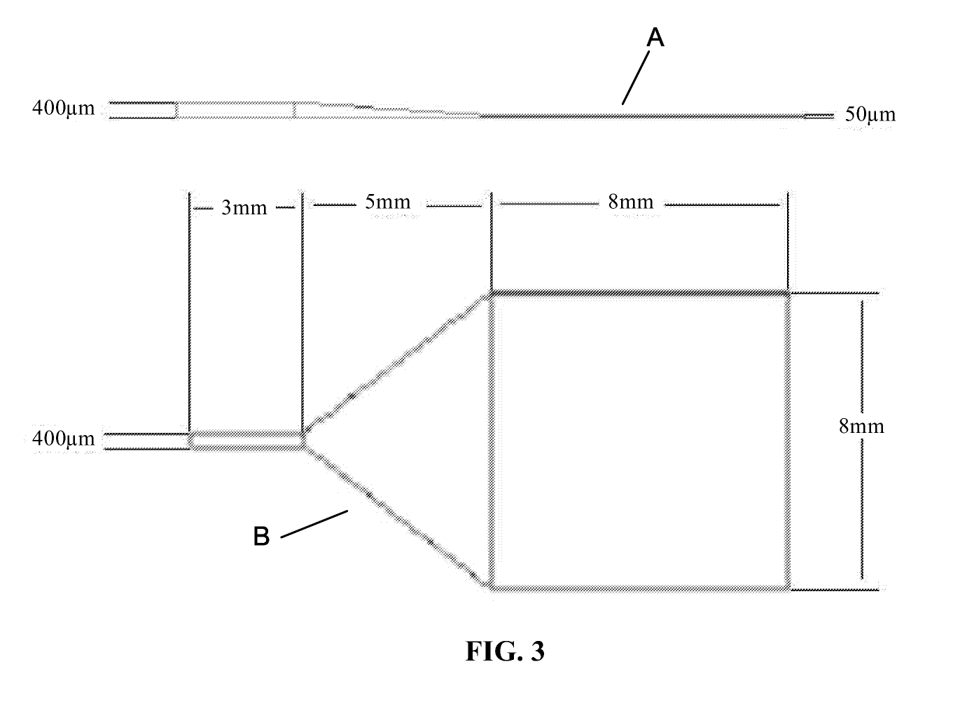

By way of example, E. coli O157:H7 cells were pre-concentrated using 1 micron diameter ferric oxide magnetic particles which were functionalized with an E. coli O157:H7 specific antibody. FIG. 2 shows a photomicrograph 20 of fluorescently labeled bacteria bound to magnetic nanoparticles; and photograph 22 of the pre-concentration subcomponent. The efficiency of capture of these bacteria by the magnetic particles in the pre-concentration subcomponent was determined using ferric oxide absorbance measurements from a spectrophotometer. The results are shown in Table 1. The samples were 0.5 mL total volumes consisting of magnetic beads linked to E. coli O157:H7 that had been pre-stained with the viability dyes. As demonstrated in FIG. 2, photograph 24, this binding was checked by pulling the magnetic beads to the side with the magnet, removing the supernatant, adding sterile water, vortexing, and then repeating the process. Alternatively, a more sophisticated flow-through/magnetic pre-capture system not requiring any manual manipulation can be used. A small volume of the sample was observed under the microscope. The fluorescence of the stained bacteria indicated a successful linkage since the beads do not fluoresce. Each sample was vortexed to create homogeneity immediately before the spectrophotometer reading was taken at an absorbance of 350 nm. The recovered samples were created by removing the supernatant liquid from the magnetic beads captured by a magnet, and then re-suspended in an equal volume of filtered, ultra pure water. For all concentrations tested, there was greater than 90% recovery. There was no indication of magnetic beads left in the supernatant fluid based on spectrophotometer readings. For larger volumes of water it is necessary to add BSA to prevent the beads from sticking to the walls of the sample tubes. This has been tested qualitatively. Magnetic beads could clearly be seen and drawn to the side of the tube in 10 mL volumes with 1% BSA, but the large amount of BSA masked the spectrophotometer readings of the re-suspended bacteria at very low concentrations of bacteria/magnetic bead complexes.

### c) Fabrication of Biosensor Array

The first step in assembling an SPR imaging system is to prepare a biosensor array with a capture ligand that specifically binds to bacteria or spores on glass slides.

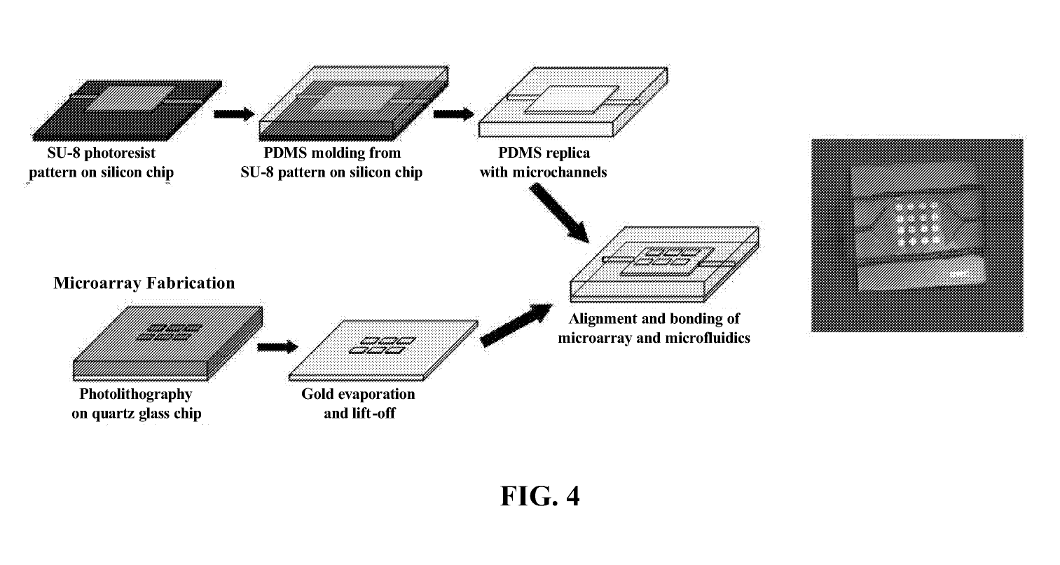

In one embodiment, glass slides can be gold-coated glass slides with a 50 nm gold film and a 2 nm-thick chromium adhesion layer. A peptide or other biomolecule pattern can be formed on the gold-coated glass using a poly(dimethyl siloxane) (PDMS) stamp. Preferably, the surface of the PDMS stamp is exposed to solutions of the inking peptide or other biomolecules (100-200 μg/ml) for 1 min. After inking, preferably the stamp is brought into contact with the gold substrate for 2 min and the gold slide is washed with a phosphate-buffered saline (PBS) solution, followed by drying with nitrogen gas. Preferably, the peptide or other biomolecule patterned gold slide is rinsed with bovine serum albumin (BSA) and Tween-20 to block nonspecific binding of bacteria. The biosensor array can be characterized by optical microscopy and tapping mode atomic force microscopy (AFM). A schematic of the microfluidic chip mold design is shown in FIG. 3 with a side view A and a top view B. The overall microfluidic chip assembly is shown in FIG. 4.

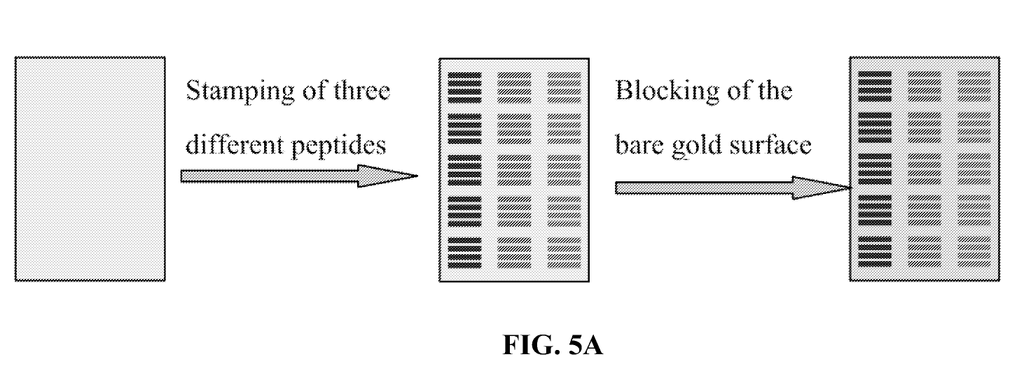

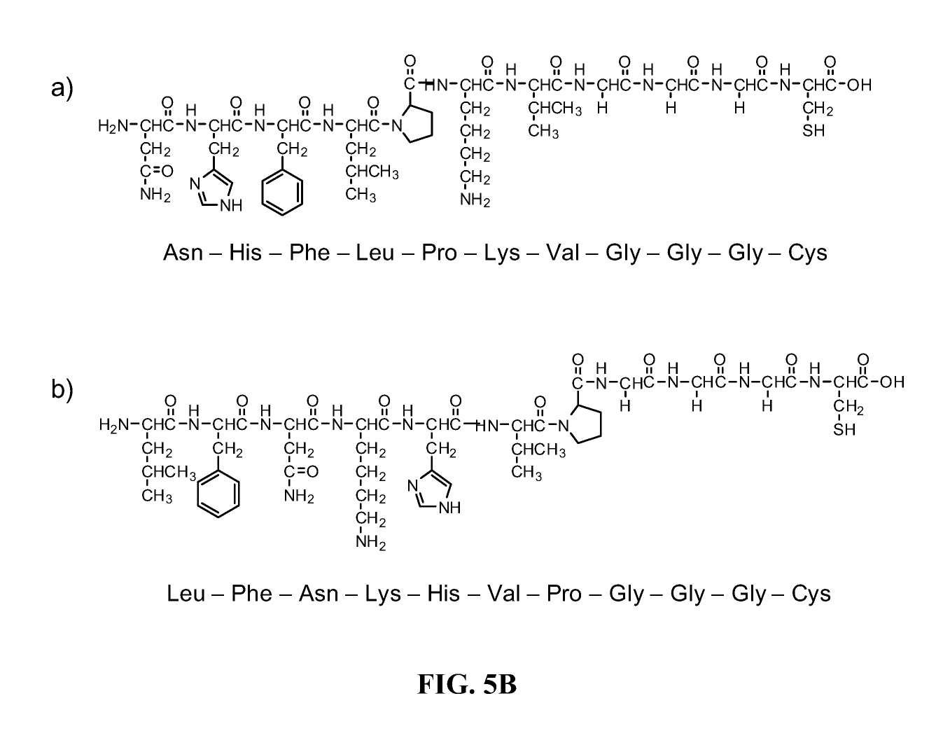

In another embodiment, there can be multiple biomolecules coupled to the sensor surface. For example, as shown in FIG. 5A, the three peptides specific to Escherichia coli O157:H7, Salmonella typhimurium, and Bacillus anthracis can be coupled to the sensor surface 50, necessitating micropatterns 52, 54, and 56 of three different peptides. Three different micropatterns on the same surface can be done by simply microcontact printing using three different PDMS stamps, each with a peptide specific to one of the bacteria. The patterned gold slide can be rinsed with bovine serum albumin (BSA) and Tween-20 to block nonspecific binding of bacteria to provide array 58.

In another embodiment, an approach for biosensor construction is the use of small molecular weight ligands that are robust to denaturation, relatively inexpensive, easily produced, and easy to modify by chemical functionalization. Recently, short peptide sequences, which specifically bind to spores of B. anthracis, have been identified by phage display peptide library screening and demonstrate exceptional selectivity in discriminating closely related Bacilli species. FIG. 5B shows two peptide sequences a and b specific towards Bacillus subtilis and Bacillus anthracis, respectively.

The peptide sequence Asn-His-Phe-Leu-Pro-Lys-Val (NHFLPKV) can be used as the binding peptide for Bacillus subtilis, and the peptide sequence, Leu-Phe-Asn-Lys-His-Val-Pro (LFNKHVP), as a specific binding peptide for Bacillus anthracis. Both peptides can be tethered to a spacer Gly-Gly-Gly-Cys (GGGC) attached to the C-terminal amino acid. Attachment of the peptide to the gold-coated sensor chip can be facilitated by a thiol-containing cysteine residue at the COOH terminal end of the peptide. In our preliminary study, peptides binding to Bacillus subtilis, Asn-His-Phe-Leu-Pro-Lys-Val (NHFLPKVGGGC), and to Bacillus anthracis, Leu-Phe-Asn-Lys-His-Val-Pro (LFNKHVPGGGC), were synthesized by standard solid-phase peptide synthesis and characterized by NMR spectroscopy, high-performance liquid chromatography (HPLC) and electrospray ionization mass spectrometry. After the successful synthesis, the peptides were micro-contact printed onto a gold-coated glass slide to generate a biosensor array and the whole array can function as multiple sensor system.

Preferably, the biosensor array will usually have microcontact printing of a linear stripe pattern instead of a solid spot. There are two reasons for this. The linear stripe pattern not only minimizes the amount of peptide required for surface grafting, but also enhances the sensitivity of detection due to close packing of the spores or cells along the stripes. Currently available SPR instruments do not measure arrays of samples, but rather measure SPR signals in independent channel(s), and therefore they lack the robust controls that array systems can deliver.

### d) Specific Capture of Pathogen on Biochip

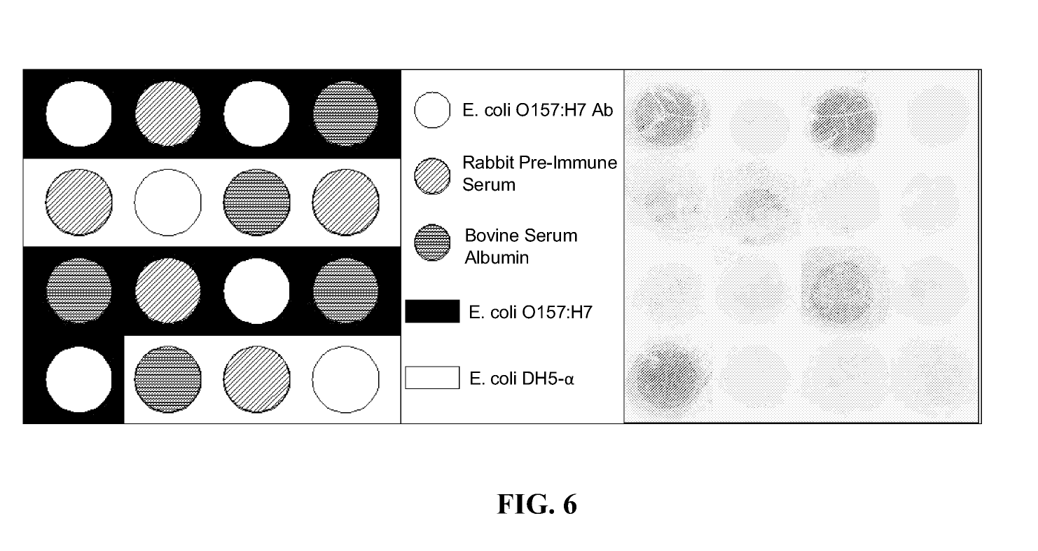

The ability to specifically capture a pathogen on a biochip was tested using fluorescence imaging. The biochip was patterned with one of three biomolecules on each gold spot. The spots were either functionalized with an E. coli O157:H7 antibody, or with one of the negative controls: rabbit preimmune serum or 1% BSA. This pattern is shown in FIG. 6. This diagram also shows which spots were exposed to E. coli O157:H7 and which ones were exposed to the negative control strain of E. coli DH5-α. To demonstrate specific capture of E. coli O157:H7, bacteria should only be present on the gold spots functionalized with E. coli O157:H7 antibodies that were exposed to E. coli O157:H7. A fluorescence image demonstrating the binding of bacteria to the array is shown in the right pane of FIG. 6. It is clear that the spots with the highest intensity are those functionalized with E. coli O157:H7 antibodies and were exposed to E. coli O157:H7.

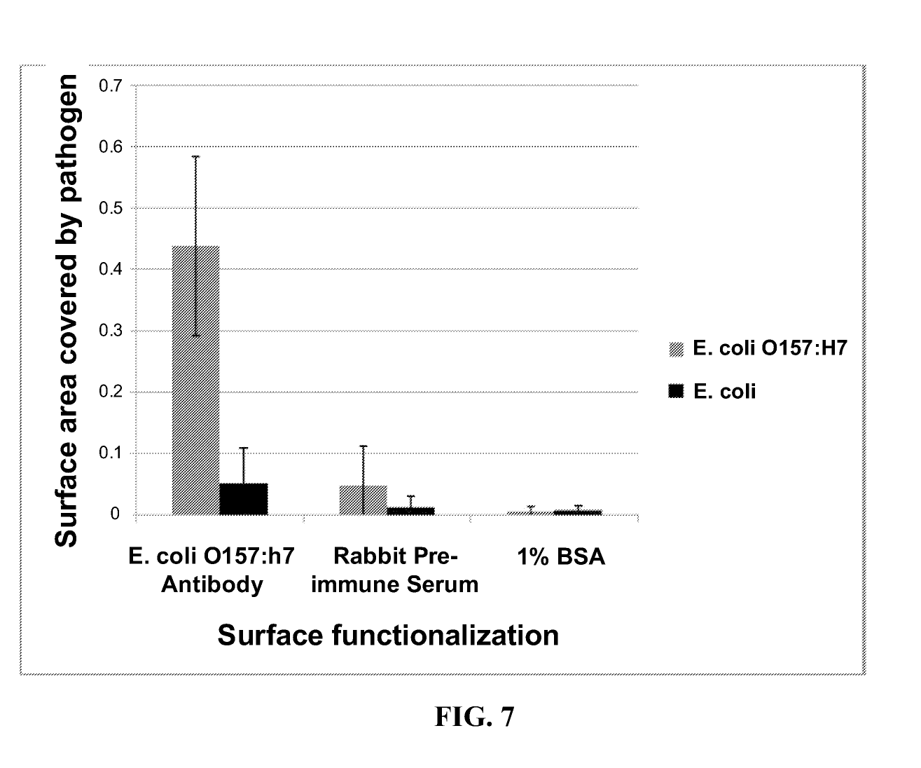

The binding of pathogen to each spot was quantified by measuring the percent of the gold spot area upon which E. coli was bound. This analysis was determined using NIH ImageJ software. The results of this analysis are shown in FIG. 7. The only conditions where a significant amount of coverage occurred were on gold spots functionalized with E. coli O157:H7 antibodies that were exposed to E. coli O157:H7, where the mean surface coverage was 43.75%. In all other cases the mean surface coverage was 5.1% or less. There was very little binding of E. coli O157:H7 to spots functionalized with rabbit pre-immune serum or BSA. As expected the E. coli DH5-α showed low levels of capture regardless of the surface functionalization. This demonstrates the specific capture of E. coli O157:H7 by antibody functionalized spots on the biochip.

### e) Surface Plasmon Resonance Imaging

SPR imaging is a sensitive, label-free method that can detect the binding of an analyte to a surface due to changes in refractive index that occur upon binding. SPR is a highly sensitive detection method which is simple, label-free, and nondestructive. SPR imaging can detect the presence of molecules or cells or pathogens bound to the biosensor surface by measuring the changes in the local refractive indices. SPR imaging involves the measurement of the intensity of light reflected at a dielectric covered by a metal (e.g., gold) layer of ˜50 nm thickness. The charge-density propagating along the interface of the thin metal layer and the dielectric is composed of surface plasmons. These surface plasmons are excited by an evanescent field typically generated by total internal reflection via a prism coupler. The wave vector of the surface plasmons is dependent upon the properties of the prism, the gold layer, and the surrounding dielectric medium (glass slide). Under appropriate conditions, the free electrons come in resonance with the incident light and a surface plasmon is generated. At this resonance condition, the reflection decreases sharply to a minimum because incident photons induce surface plasmons instead of being reflected. Changes in dielectric properties, e.g., thickness or refractive index, of the surrounding medium lead to changes in the wave vector and consequently there is a shift of plasmon resonance minimum of the reflected light.

The adsorption or recognition of biomolecules, bacteria, or cells is accurately detected, as the plasmon resonance is extremely sensitive to dielectric properties and the fact that resonance occurs only in a small range (either wavelength or angle of incidence). Resonance angle measurements have been used for chemical and biochemical sensing. Only p-polarized light in plane of incidence with the electric field vector oscillating perpendicular to the plane of the metal film is able to couple to the plasmon mode. The s-polarized light, with its electric field vector oriented parallel to the metal film, does not excite plasmons. Since s-polarized light is reflected by the metal surface, it can be used as a reference signal to improve the sensitivity. In SPR imaging, the reflectivity change resulting from biomolecular and cellular binding on the biosensor surface is measured. The reflectivity change, Δ% R, is determined by measuring an SPR signal at a fixed angle of incidence before and after analyte binding. The SPR imaging setup captures data for the entire probe array, including controls to detect non-specific binding as described later in this proposal, simultaneously on a charge coupled device (CCD) camera. Surface plasmon resonance imaging can be used to measure simultaneous binding events on microarrays.

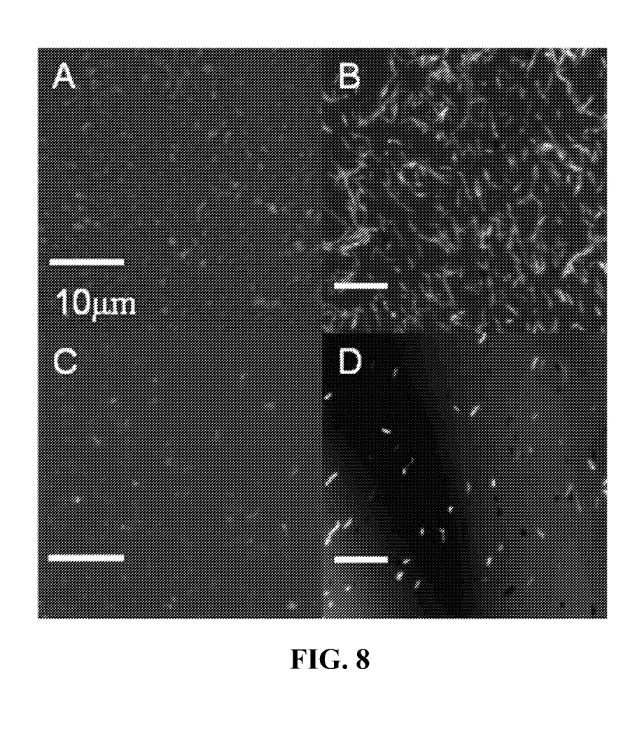

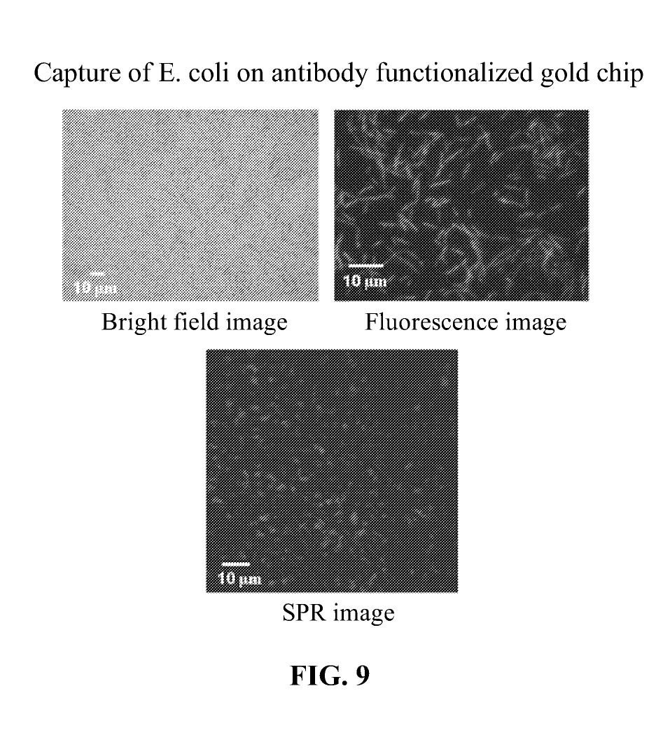

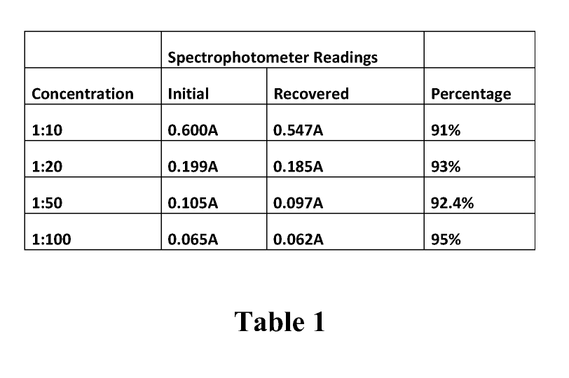

In one example, a bench top SPR imaging system was used to take several SPR images of E. coli bound to a gold coated slide. Examples of these SPR images at areas of different E. coli densities are contained in FIG. 8 and FIG. 9. These figures also contain epi-fluorescence images of the bacteria at corresponding densities to the SPR images. The SPR images and epi-fluorescence images are not of the same field of view. Single pathogens were successfully imaged using SPR and epi-fluorescence imaging. Even if the fields of view were the same, SPR images only show the points where the bacteria is in contact (within surface plasmon resonance distance and conditions) with the gold surface. Hence SPR images only partially correlate with the epi-fluorescence images because the latter represents a top view of all bacteria, whether or not they are within SPR imaging distance/conditions of the surface.

In another embodiment, a portable hybrid imaging unit can be used to detect pathogens. Preferably, the system is made portable using a battery powered high output light-emitting diode for epi-fluorescent illumination and a battery powered laser diode for surface plasmon resonance illumination. The system can also be made portable using a compact rigid optical cage construction to eliminate degrees of freedom of motion. Preferably, the cage construction keeps the illumination aligned through the optical axis, even if the device is moved. More preferably, the surface plasmon resonance imaging and detection angles are made adjustable, because of the hinged nature of the optical cage construction, so as to optimize the device to experimental conditions. In particular, the incidence angle can be optimized for different types of assays or different chip types. The hinge occurs at the SPR prism, which acts as a fixed point for the mounting of the system inside a protective case, allowing for portability.

### Examples

### Example 1

### Bacterial Strains, Growth and Staining

Two strains of E. coli, pathogenic E. coli O157:H7 (Castellani and Chalmers strain, ATCC, Manassas, Va.) and the nonpathogenic E. coli DH5-α, (provided by Arthur Aronson, PhD, Dept. of Biological Sciences, Purdue University, West Lafayette, Ind.) were used for proof-of-concept experiments. The bacteria were streaked onto an LB (Luria-Bertani) plate and incubated at 37° C. overnight. Single isolated colonies were aseptically harvested from the LB plate and allowed to grow in 10 mL of LB broth overnight.

In order to assess the fraction of bacterial cells of each strain a simple fluorescence method live/dead bacteria determinations was used. BacLight™ Bacterial Viability Kits (Invitrogen, Inc., Carlsbad, Calif.) provides a sensitive, single-step, fluorescence-based assay for bacterial cell viability. Importantly these well-established assays can be completed in minutes and do not require wash steps. The assays work on bacterial suspensions or bacteria trapped on peptide arrays and are well-suited for subsequent detection by simple fluorescent imaging. There is no need to resolve or count individual bacteria. We merely need to get a categorical level of fluorescent intensity on the array. The LIVE/DEAD BacLight Bacterial Viability Kits employ two nucleic acid stains—the green-fluorescent SYTO® 9 stain and the red-fluorescent propidium iodide (PI) stain. Both of these dyes have extremely low quantum efficiencies unless bound to nucleic acids, so background fluorescence is extremely low and there is no need for any wash steps. These stains differ in their ability to penetrate healthy bacterial cells. When used alone, the SYTO 9 stain labels both live and dead bacteria. In contrast, PI penetrates only bacteria with damaged membranes, reducing SYTO 9 fluorescence when both dyes are present. This is achieved both by competition and by fluorescent donor quenching if in sufficiently close proximity to have energy transfer taking place between the SYTO 9 and the PI. Thus, live bacteria with intact membranes fluoresce green, while dead bacteria with damaged membranes fluoresce red. Live and dead bacteria can be viewed separately or simultaneously by fluorescence microscopy with suitable optical filter sets.

### Example 2

### Magnetic Pre-Concentration

Magnetic pre-concentration was accomplished using superparamagnetic 1 μm iron oxide beads (Bang's Labs, Fishers, Ind.) coupled with antibodies specific to a membrane antigen on E. coli O157:H7. This linked the bacteria to one or two magnetic beads. After washing with water, the coupled beads and bacteria were diluted with water into different concentrations from 1:10 to 1:100 with a total volume of 0.5 mL. Each of these concentrations was measured in a UV-Vis spectrophotometer (Genesys 10 uv, Thermo-Fisher, Waltham, Mass.) at 350 nm, which is a wavelength absorbed by iron oxide. Next a 200 mT magnet was used to draw the magnetic beads to the side of the tube so that the supernatant fluid could be removed. Previous experiments have shown us that 200 mT is sufficient to recover the magnetic beads. An equivalent amount of water was then added to the beads and shaken. The absorbance at 350 nm of the re-suspended bead mixture was then measured in the spectrometer. The supernatant fluid was also measured in the spectrophotometer to check for stray magnetic beads to help determine the capture efficiency.

### Example 3

### Microfluidic Chip Assembly

The microfluidic chip was designed using Ansoft HFSS v10.1 software (Ansoft, Pittsburgh, Pa.). The resin mold (Accura SI 10 polymer, 3D Systems Corp., Rock Hill, S.C.) for this chip was then created using a stereo lithography machine (VIPER si2T SLA System by 3D Systems). Once the mold was cured with UV light, a 1:10 ratio of curing agent to PDMS polymer was mixed and then poured over the mold. This was allowed to cure overnight. Next, the PDMS was peeled off the resin mold an inlet port was punched using a blunt tipped 28 gauge needle. Next, the PDMS was attached to a clean glass slide using a Corona plasma etch system (BD 20AC, Electro-Technic Products Inc., Chicago, Ill.). The Corona system is a handheld device that creates a localized plasma field at room temperature and can oxidize the PDMS surface. This was used to treat the PDMS for approximately 20 seconds and then the PDMS was pressed onto the glass slide and heated on a hotplate at 70° C. for 15 minutes to ensure a good seal. The Corona process is important because it does not require higher temperatures that may damage antibodies, peptides, or other capture molecules during the process of bonding the microfluidic structure to the gold contact-printed slide. After this tubing was inserted into the port and sealed with uncured PDMS.

### Example 4

### Specific Pathogen Capture on Biochip

The base chip used was a glass slide with a 4×4 array of 1 mm diameter gold spots (GWC Technologies, Madison, Wis.). The surface of the chip was cleaned by immersion in a 1:1 mixture of sulfuric acid and 30% hydrogen peroxide. This will remove any organic matter from the surface of the biochip, as well as expose free electrons on the gold surface for biomolecule attachment. Three biomolecules were used to functionalize the gold spots. The first was an antibody that specifically binds E. coli O157:H7. The second was rabbit pre-immune serum, which is a negative control. The third was 1% bovine serum albumin solution in water (BSA, Sigma-Aldrich, St. Louis, Mo.) that is a second negative control. The array was patterned by applying 1 μL (at a concentration of 100 mg/mL) of a treatment to each gold spot. Each gold spot received only one treatment, which was left to adsorb to the surface for one hour at room temperature. The chip was then washed with phosphate buffered saline (PBS), and then 1% BSA to occupy any remaining active sites on the gold surface, as well as non-specific sites on the antibodies. Two strains of E. coli, E. coli O157:H7 and E. coli DH5-α were then selectively introduced to the array. Each strain was fluorescently labeled with Syto-9 dye (Invitrogen Inc., Carlsbad, Calif.). The bacteria were allowed to incubate at room temperature for 10 minutes, and unbound bacteria were washed away with PBS.

The capture of the bacteria was assessed using epi-fluorescence microscopy (Nikon Diaphot Inverted Fluorescence Microscope, Nikon Inc., Melville, N.Y.). A fluorescence image of each spot was captured, and the presence of captured pathogen was quantified by image analysis using NIH ImageJ software (http://rsbweb.nih.gov/ij/). The percentage of the surface area of each gold spot covered by a pathogen was calculated by applying a threshold to each pixel, pixels covered by a pathogen had an intensity above the threshold. The surface area coverage was then determined by dividing the number of thresholded pixels from the total number of pixels in a gold spot.

### Example 5

### Construction of Bench Top Surface Plasmon Resonance Imaging System

A bench-top surface plasmon resonance imaging system was built based on the Kretschmann configuration, whereby a thin gold film is directly deposited on a slide sitting on top of the prism that is used to generate the necessary evanescent wave at the metal-dielectric interface by means of total internal reflection. The device was constructed on an optical breadboard using post mount optics. An inexpensive 635 nm laser diode (Edmund Optics, Barrington, N.J.), was used to illuminate the sample, which is placed on top of a SFL111 equilateral prism (Edmund Optics, Barrington, N.J.). The prism is mounted on a goniometer (Thorlabs, Newton, N.J.) which is used to control the incidence angle of the laser. An inexpensive computer controlled CCD camera (Pt. Gray Research, Richmond, BC, Canada) is then used to collect the SPR image.

### Example 6

### Design and Construction of the Portable Hybrid Imaging System

A more portable hybrid imaging system was constructed. This prototype utilizes the Microptic optical cage system (AF Optical, Fremont, Calif.) to make a three armed device. The SPR arms are based on the Kretschmann configuration. A BK7 glass right angle prism (Thorlabs, Newton, N.J.), is mounted at the center of the three arms. The prism mounts contain variable angle slots, which allow the SPR illumination arm and detection arm to swing to create the appropriate incident angle. The SPR illumination arm consists of a 635 nm diode laser (Thorlabs, Piscataway, N.J.) that is then shaped by a beam expander to illuminate the whole sample. A polarizer on a rotary mount (AF Optical, Fremont, Calif.) is used to generate p-polarized light. The SPR detection arm consists of a 4× long working distance objective (Olympus), a focusing lens and a CCD camera (Pt. Gray Research) to capture the SPR image. The epi-fluorescence imaging arm uses a 4× objective to image the sample, with the standard excitation (480/20 nm band pass) dichroic (500 nm long pass dichroic) and emission filter setup (515/20, or 565/30 nm band pass). An ultra-bright 470 nm LED is used to illuminate the sample (LumiLEDs, San Jose, Calif.) for molecular imaging of the fluorescently stained bacteria and a CCD camera (Pt. Gray Research) is used to image the sample. Both cameras are connected to a notebook computer (Dell Inspiron 1300, Dell Computers, Round Rock, Tex.) where frame grabber software acquires the images (PixelScope Pro, Wells Research Co., Lincoln, Mass.). The microfluidic chip was placed on top of the prism where it can be imaged by both SPR imaging and epi-fluorescence molecular imaging.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

We claim:

1. A sensing system for the detecting biological agents, comprising:
a pre-capture unit adapted to sequester pathogens from a fluid or gas and increase pathogen concentration into a volume suitable for a microfluidic biochip unit;
a microfluidic biochip unit coupled to the pre-capture unit, the microfluidic biochip having contact printed surfaces comprising pathogen-specific capture ligands adapted to capture pathogens;
a surface plasmon resonance imaging unit adapted to detect the captured pathogens by surface plasmon resonance imaging;
a molecular imaging unit adapted to detect the captured pathogens by epi-fluorescence imaging; and
at least one small imaging camera adapted to capture surface plasmon resonance and molecular imaging data, the at least one small imaging camera coupled to a computing device.

2. The sensing system of claim 1 wherein the pre-capture unit is adapted to capture magnetic micro- or nanoparticle labeled microbes.

3. The sensing system of claim 1 wherein the contact printed surfaces comprise gold.

4. The sensing system of claim 1 wherein the pathogen-specific capture ligands comprise at least one of peptides, antibodies, and aptamers.

5. The sensing system of claim 2 wherein the magnetic micro- or nanoparticle labeled microbes are coated with at least one of peptides, antibodies, and aptamers.

6. The sensing system of claim 2 wherein the magnetic micro- or nanoparticle labeled microbes are coated with lipophilic molecules.

7. The sensing system of claim 1 wherein the system is portable.

8. The sensing system of claim 1 wherein the at least one small imaging camera is a high resolution digital camera for real time imaging of pathogenic bacteria and spores that become bound to the sensor surface.

9. The sensing system of claim 1 wherein the system is adapted to simultaneously detect the presence of more than one type of pathogen.

10. The sensing system of claim 1 wherein the computing device performs automated image analysis.

11. The sensing system of claim 1 wherein the computing device is configured to automated analysis for pathogen detection.

12. A sensing system for the detection of biological agents, comprising:
a hybrid microfluidic biochip adapted to perform multiplexed detection of single celled pathogens using a combination of surface plasmon resonance and epi-fluorescence imaging.

13. A method for the detection of biological agents, comprising the steps of:
a) concentrating a biological sample into a smaller volume suitable for a microfluidic flow/imaging device;
b) flowing the concentrated sample through a microfluidic unit having contact printed surfaces comprising pathogen-specific capture ligands;
c) detecting captured pathogens with a surface plasmon resonance unit;
d) detecting captured pathogens with a molecular imaging unit; and
e) collecting surface plasmon resonance and molecular imaging data with at least one small imaging camera and a computing device.

14. The method of claim 13 wherein a magnetic field is employed to concentrate the sample, the sample comprising cells bound to magnetic microspheres.

15. The method of claim 14 wherein the sample is concentrated by the steps of:
a) introducing a flow of the sample to the magnetic field;
b) trapping cells bound to magnetic microspheres in the magnetic field;
c) removing cells and sample not trapped in the magnetic field;
d) removing the magnetic field so as to release the trapped cells bound to magnetic microspheres; and
e) transporting the cells bound to magnetic microsphere with a small amount of fluid to the microfluidic unit.

16. The sensing system of claim 7 wherein the system comprises a battery powered high output light-emitting diode for epi-fluorescent illumination.

17. The sensing system of claim 7 wherein the system comprises a battery powered laser diode for surface plasmon resonance illumination.

18. The sensing system of claim 7 wherein the system comprises a compact rigid optical cage construction to eliminate degrees of freedom of motion.

19. The sensing system of claim 7 wherein the system comprises a cage construction adapted to maintain illumination alignment through an optical axis.

20. The sensing system of claim 7 wherein surface plasmon resonance illumination angles and detection angles are adjustable.

21. The sensing system of claim 1, wherein the system is adapted to detect the live/dead status of at least one type of pathogen.

22. The sensing system of claim 1, wherein the system is adapted to detect the metabolic status of at least one type of pathogen.
